(19) United States
(12) Patent Application Publication
(10) Pub. No.: US 2005/0148519 A1
(43) Pub. Date: Jul. 7, 2005

(54) Salts of pharmacologically active compounds

(76) Inventor: Yerramilli V. S. N. Murthy, Apex, NC (US)

Correspondence Address: MORGAN LEWIS & BOCKIUS LLP, 1111 PENNSYLVANIA AVENUE NW, WASHINGTON, DC, 20004, US

(21) Appl. No.: 10/974,833

(22) Filed: Oct. 28, 2004

Related U.S. Application Data
(60) Provisional application No. 60/515,967, filed on Oct. 29, 2003.

(51) Int. Cl. A61K 31/7048; A61K 31/7052; A61K 31/192; A61K 31/195; A61K 31/65

(52) U.S. Cl. 514/28; 514/567; 514/35; 514/29; 514/152; 514/569; 514/570

(57) ABSTRACT

The present invention relates to compositions containing at least two pharmacologically active ingredients. The compositions comprise a proton-donating pharmacologically active ingredient and a proton-accepting pharmacologically active ingredient in the form of a neutral salt. The salt can be dissolved in a solvent. Also provided are methods of administering pharmacologically active ingredients and methods of treating a disorder in an animal comprising administering to an animal in need thereof a salt of the invention.

### CROSS REFERENCE TO RELATED APPLICATIONS

This application claims the benefit of U.S. Provisional Application No. 60/515,967, filed Oct. 29, 2003, the contents of which are expressly incorporated herein by reference.

### STATEMENT REGARDING FEDERALLY SPONSORED RESEARCH OR DEVELOPMENT

Not Applicable.

### INCORPORATION BY REFERENCE TO MATERIAL SUBMITTED ON A COMPACT DISC

Not Applicable.

### FIELD OF THE INVENTION

The present invention relates to compositions comprising a salt formed from at least two pharmacologically active ingredients and methods of treating a disorder in an animal comprising administering to an animal in need thereof a salt of the invention.

### BACKGROUND OF THE INVENTION

The following discussion of the background of the invention is merely provided to aid the reader in understanding the invention and is not admitted to describe or constitute prior art to the present invention.

Bacterial infections in animals (human and non-human) often result in severe pain followed by elevated body temperatures. Treatment of bacterial infections generally includes the administration of anti-infectives and antimicrobials along with anti-inflammatories to control the pain and reduce elevated temperatures. Generally, anti-infective compositions treat diseases caused by bacteria, and have limited or no utility with viruses or protozoa. These compositions are further categorized as antibiotics and antimicrobials. Anti-microbials generally have biological activity against protozoal, viral, or fungal pathogens but can also have activity against bacterial pathogens.

Known antibiotics include, for example, macrolides such as azithromycin, roxythromycin, tilmicosin, tetracyclines such as oxytetracycline and doxycycline, fluoroquinolones such as enrofloxacin, and β-lactams such as cephalosporins and penicillins and aminoglycosides.

Known non-steroidal anti-inflammatories (NSAIDs) include, for example, flunixin, carprofen, ibuprofen, naproxen and ketoprofen. A disadvantage of these compounds is that they are cleared from the patient's system relatively rapidly (i.e., they have short half-lives) and generally require multiple daily dosages in order to attain therapeutic effectiveness. For example, mastitis in cattle is treated with anti-infectives (single or multiple doses of appropriate antibiotic such as tilmicosin, oxytetracycline, doxycycline) accompanied by daily injections of flunixin (an anti-inflammatory) for 3-4 days. Immediately following administration of a single dose of flunixin (at 1.1 mg/kg dose), the serum concentrations rise to anywhere between 8-13 μg/ml but rapidly drop to sub micro gram/mL within 4 hours of administration and undetectable levels 6-12 hours post administration.

Medications, such as anti-inflammatories, are often concomitantly administered with anti-infectives to reduce suffering attributable to trauma and pain during pre and post surgical conditions. Many of these compounds are potent cyclooxygenase inhibitors and thus block the synthesis of prostaglandin. Prostaglandins play a cytoprotective role in gastric mucosa by inhibiting proton pumps and thereby decreasing gastric acid production. They also promote the generation of a protective barrier of mucous and bicarbonate. Inhibition of prostaglandin synthesis by medication may produce gastrointestinal ulceration. Dog studies have shown, for example, that vomiting, diarrhea, blood in stools, and gastro-intestinal ulceration were common following oral dosing with 2.2, 6.6 and 11.0 mg/kg of the anti-inflammatory flunixin.

Certain medical compounds produce injection site reactions. Flunixin, for example, can cause tissue damage when this drug is administered either intramuscularly or subcutaneously, and thus is generally recommended to be administered intravenously. Oxytetracyclines can cause severe injection site inflammations and even necrosis, and tilmicosin can cause mild injection site reactions that can take two to three days to dissipate.

It would be advantageous if a less frequent or even “single dose” of a pharmaceutical formulation could provide a complete regimen of both antimicrobial and anti-inflammatory drugs in a controlled manner over the required period of time. It would also be advantageous if such formulations could be used without the negative side effects associated with the administration of either the anti-infective or anti-inflammatory alone.

### SUMMARY OF THE INVENTION

The invention relates to compositions comprising a salt formed from two or more pharmacologically active ingredients. The pharmacologically active ingredients are combined to form a salt based on ionic attractions. The salt is formed from a proton-accepting (i.e., “basic”) pharmacologically active ingredient and a proton-donating (i.e., “acidic”) pharmacologically active ingredient. In one embodiment, the salts are formed by combining a proton-accepting or basic antibiotic (e.g., azithromycin, roxythromycin, tilmicosin, oxytetracycline and doxycycline) with a proton-donating or acidic anti-inflammatory (e.g., flunixin, carprofen and naproxen). Formation of the salts do not involve any chemical modification of the structure of the pharmacologically active ingredient, other than formation of the salt. In one embodiment, the salt is a solid. In another embodiment, the salts are dissolved in a pharmaceutically acceptable solvent, such as a water miscible organic solvent (e.g., propylene glycol, glycerol formal, N-methyl pyrrolidone (NMP), ethanol, and polyethylene glycol (PEG)), or a water immiscible solvent (e.g., isopropyl myristate, ethyl lactate, castor oil, safflower oil and soybean oil). The compositions containing the salt and pharmaceutically acceptable solvent can be true, injectable solutions, but suspensions and other means of delivery are contemplated, such as topical, oral, or nasal delivery. The pharmacologically active ingredients typically have a different net electronic charge when in a “free” or unbound form compared to the salt form. At least one of the pharmacologically active ingredients is a proton-donor and at least one is a proton-acceptor.

Thus, in a first aspect, the present invention provides compositions containing a salt of a proton-donating pharmacologically active ingredient and a proton-accepting pharmacologically active ingredient. In one embodiment, the proton-donating pharmacologically active ingredient has anti-inflammatory activity. In various embodiments, the pharmacologically active ingredients can be anti-infectives, anti-microbials, or antibiotics. In one embodiment, the proton-donating pharmacologically active ingredient is bound to the proton-accepting pharmacologically active ingredient through an ionic attraction.

In various embodiments, the proton-donating, pharmacologically active ingredient is a non-steroidal anti-inflammatory (NSAID) such as, for example, flunixin, carprofen, naproxen, ibuprofen, diclofenac, or ketoprofen; the proton-accepting pharmacologically active ingredient is an antibiotic such as, for example, azithromycin, roxythromycin, tilmicosin, oxytetracycline or doxycycline. In one embodiment of the invention, the proton-donating pharmacologically active ingredient is flunixin and the proton-accepting pharmacologically active ingredient is tilmicosin. The composition can be provided in a pharmaceutically acceptable carrier as an injectable composition or as a suspension. In a preferred embodiment, the composition further comprising a pharmaceutically acceptable organic solvent precipitates when injected into water. The injectable composition can be a true solution. In various embodiments, the composition is provided as a liquid, a suspension, or a solution form. The composition can also be provided as a solid (e.g., a crystal) or as an injectable formulation. Tilmicosin-flunixin is an example of a salt of the invention.

By “injectable formulation” or “injectable composition” is meant a formulation or composition that can be injected, i.e., drawn into a syringe and injected subcutaneously, intraperitoneally, or intramuscularly into an animal without causing adverse effects due to the presence of solid materials in the composition. Solid materials include, but are not limited to, crystals, a gummy mass, and a gel.

The term “suspension,” as used herein, means solid particles that are evenly dispersed in a solvent, which can be aqueous or non-aqueous. In one embodiment, the particles have an average particle size of less than about 100 μm determined using a particle size analyzer such as commercially available from Microtrac Inc. of Montgomeryville, Pa.

By “pharmacologically active” is meant that the compound or ingredient causes a pharmacological effect in the treated animal. For example, the effect may be to destroy, hinder, or prevent growth of bacteria, parasites, or fungi in the treated animal, or to reduce inflammation in a tissue of the animal, or another pharmacological, therapeutically significant and measurable effect in the treated animal, and have a reasonable benefit/risk ratio. A reasonable benefit/risk ratio refers to a significant benefit being obtained by use of the compound (e.g., effective treatment of a disease or condition requiring need for treatment) with a small or minimal and medically acceptable risk (i.e., low incidence and severity of significant and negative effects associated with use of the compound). For the purposes of this definition, inorganic ions (e.g., Na, Cl, Mg, Mn) are not pharmacologically active as they are normally not therapeutically useful and do not have a pharmacologic effect in a treated animal.

By “water miscible” is meant that the solvent is capable of mixing in any ratio in water without separation of two phases. By “water soluble” is meant that the solvent has some significant level of solubility in aqueous solutions, e.g., triacetin is considered a water soluble solvent since it is soluble in water at a ratio of up to about 1:14. By a “true solution” is meant a solution having substantially no suspended particulate matter. By “substantially no suspended particulate” is meant that no more than 10% of the formulation is retained on a 0.22 μm filter when the formulation is filtered through the filter at 98° F. at

In other embodiments, the proton donating or proton accepting pharmacologically active ingredient is a COX-2 inhibitor such as, for example, celecoxib, or is a macrolide, a tetracycline, a doxycycline, a fluoroquinolone such as enrofloxacin, beta-lactams such as cephalosporins, penicillins, an aminoglycoside, or an anti-fungal (e.g., terbinafine). Other molecules that can be used as the proton-donating or accepting pharmacologically active ingredients include the NSAIDs phenylbutazone, tolfenamic acid, diclofenac, and vedaprofen.

In another embodiment, the compositions further comprise a salt made from a proton-donating pharmacologically active ingredient or a proton accepting pharmacologically active ingredient and a lipophilic counterion, i.e., a counter ion derived from a lipopohilic molecule. The lipophilic counterion can be, for example, the anion of a saturated or unsaturated fatty acid of any specific number of carbons between 8 and 22, such as 8, 9, 10, 11, 12, 13, 14, 15, 16, 17, 18, 19, 20, 21, or 22 carbons. Representative C8-C₂₂ fatty acids include, but are not limited to, caproic acid, lauric acid, myristic acid, palmitic acid, stearic acid, palmic acid, oleic acid, linoleic acid, and linolenic acid. In one embodiment, the fatty acid is a C₈-C₁₈ fatty acid and in another embodiment a C₁₀-C₁₈ fatty acid, such as lauric acid, linoleic acid, decanoic acid, myristic acid, or oleic acid. Other lipophilic acids may also be used, for example dicarboxylic acids, lipophilic poly-carboxylic acids, and aromatic acids. A representative dicarboxylic acid is sebacic acid. A representative aromatic acid is benzoic acid. Representative poly-carboxylic acids include, but are not limited to, polyaspartic acid, polyacrylic acid, polysebacic acid, polybenzoic acid, or combinations thereof.

Basic lipophilic molecules can also be used to form the lipophilic counter ion (i.e., by being protonated by an acidic pharmacologically active ingredient). Representative basic lipophilic molecules include, but are not limited to, sphingomyelins and long chain aliphatic amines (e.g., amines having between 8 and 22 carbons).

In still other embodiments, mixtures of any of the salts described herein can be provided in the compositions.

Thus, compositions are contemplated including a salt of a proton donating and a proton accepting pharmacologically active ingredient and also containing one or more salts of a proton donating or proton accepting pharmacologically active ingredient with a lipophilic counterion, and combinations thereof. By a “lipophilic counterion” is meant an ionic form of a fat soluble molecule. The lipophilic counterion may be an anion of a fatty acid, but may also be another fat soluble molecule, such as a protonated long chain aliphatic amine. The lipophilic molecule can be a proton donor or a proton acceptor. The particular water/octanol partition coefficient of a lipophilic molecule will vary. In one embodiment the lipophilic molecules have a water/octanol partition coefficient of 100 or greater. In other embodiments the coefficient is 50 or greater (e.g., benzoic acid), or 40 or greater, or 25 or greater, or 10 or greater.

In one embodiment, the proton donating pharmacologically active ingredient is flunixin and the proton accepting pharmacologically active ingredient is tilmicosin. Tilmicosin, however, has two basic amine sites and therefore can form a salt with two molecules of flunixin. Often, however, it is desirable to have a salt formulation according to the invention having less than 2 equivalents of flunixin for each equivalent of tilmicosin. When less than two equivalents of flunixin are used to form the tilmicosin-flunixin salt (e.g., 1 equivalent of flunixin and 1 equivalent of tilmicosin) then some molecules of tilmicosin will be protonated twice and other molecules if tilmicosin will not be protonated at all, in other words not all the tilmicosin molecules are mono-protonated. In this case, the unprotonated tilmicosin molecules can be released from a dosage formulation more rapidly than is desirable. To prevent or control this a lipophilic acid, e.g., a fatty acid, is used to protonate some of the tilmicosin molecules. For example, the salt may comprise 1 equivalent of flunixin, 1 equivalent of a fatty acid, and 1 equivalent of tilmicosin. This will assure that every molecule of tilmicosin is protonated at both basic sites.

In another aspect the present invention provides methods of treating a condition in an animal comprising administering a pharmacologically active composition to an animal. The methods involve administering to the animal a composition of the invention, as described above. In one embodiment, solid compositions are administered by implanting the solid under the skin of the animal. The composition further comprising a pharmaceutically acceptable organic solvent can be administered by injection. In another embodiment, the proton-donating pharmacologically active ingredient and the proton-accepting pharmacologically active ingredient have slower release kinetics in the animal when administered as a salt according to the present invention than when administered in free form. In yet another embodiment, the composition further comprising a pharmaceutically acceptable organic solvent is injected to form a drug depot in the animal that releases the pharmacologically active ingredient(s) over time into the blood or tissues of the animal. The “free form” refers to the non-ionic form of the pharmacologically active ingredient.

By “salt” is meant two compounds that are chemically bound by an ionic attraction. The attraction may also be the result of a combination of an ionic bond and a hydrogen bond, and may even have partial covalent properties. Thus, for example, a salt of flunixin and tilmicosin refers to flunixin bound to tilmicosin through an ionic attraction. With respect to this definition, it is understood by persons of ordinary skill in the art that chemical bonds are often not exclusively covalent nor exclusively ionic. Thus, when a bond (or attraction) is referred to as “ionic” it is meant that at least 90% of the attractive force between the bonded species results from an ionic attraction. In one embodiment, preferably at least 95%, more preferably at least 97%, or most preferably at least 99% of the attractive force between the bonded species results from an ionic attraction. When a bond is referred to as “covalent” it is meant that at least 90% of the attractive force between the bonded species results from a covalent interaction. In one embodiment, preferably at least 95%, more preferably at least 97%, or most preferably at least 99% of the attractive force between the bonded species results from a covalent attractions. By “positively charged” is meant that a molecule or pharmacologically active ingredient has a net positive charge. By “negatively charged” is meant that a molecule or pharmacologically active ingredient has a net negative charge. Although various molecules may have a portion of the molecule having a positive charge or a negative charge, the definitions for “positively charged” and “negatively charged” are meant to refer to the molecule as a whole. By “acidic” is meant a form of a compound that is a proton donor. By “basic” is meant a form of a compound that is a proton acceptor. By a “proton donor” is meant an ion or molecule that can lose an H⁺ ion or proton (also sometimes referred to as a Bronsted acid). By a “proton acceptor” is meant an ion or molecule that can gain an H⁺ ion or proton (also sometimes referred to as a Bronsted base). The proton donor and a proton acceptor can form a salt and be bound by ionic attractions. By “bound” is meant that the members are held together by a type of chemical bond, whether covalent, ionic, or H-bond.

By “anti-infective” is meant a chemical that acts against infection by inhibiting the spread of an infectious agent or by killing the infectious agent outright. Anti-infective is a general term that encompasses antibacterials, antibiotics, antifungals, antiprotozoans and antivirals. By “anti-microbial” is meant a chemical that destroys or inhibits the growth of microorganisms. An “antibiotic” is an antimicrobial agent made from a mold or a bacterium that kills or slows the growth of other microbes, specifically bacteria. Examples include penicillin, streptomycin, azithromycin, roxythromycin, tilmicosin, oxytetracycline, and doxycycline. An “anti-fungal” is a chemical that destroys or hinders the growth of one or more fungi.

By “precipitate” is meant a substance separated from a solution or suspension as an insoluble solid.

A “pharmaceutically acceptable solvent” is a liquid that dissolves a salt of the invention and that is suitable for use with humans and/or animals without undue adverse side effects (such as toxicity, irritation, and allergic response) and commensurate with a reasonable benefit/risk ratio.

The term “release kinetics” refers to the time course in which pharmaceutically active molecules are released into the blood or tissues of an animal.

By “drug depot” is meant a concentration or precipitation of a pharmacologically active ingredient within the body of the treated animal that releases a pharmaceutically effective amount of the active compound over time. By “pharmaceutically effective amount” is meant an amount that exerts a measurable and medically significant effect on the treated animal, resulting in progress towards curing, arresting, or preventing the subject disease, or alleviating or preventing the condition that was the reason for treatment.

In various additional aspects, the invention provides methods of treating pain in an animal, methods of treating inflammation in an animal, methods of administering antibiotics to an animal, methods of administering anti-infectives to an animal, methods of treating a bacterial infection in an animal, and methods of treating a fungal infection in an animal (e.g., the lung). All of these methods are accomplished by administering a composition of the invention to the animal. The mode of administration can be any form of injection, such as sub-cutaneous, sub-dermal, intra-peritoneal, intra-pleural, and other forms of injection. The pharmaceutical compositions can also be administered topically, orally, or nasally. In one embodiment, the bacterial or fungal infection is an infection of the lung. In various embodiments, the animal can be a human, a canine, a feline, an equine, a bovine, an ovine, a porcine, an amphibian, a reptile, or an avian. In one embodiment, the animal is a mammal. In one embodiment, the animal is a human, a canine, a feline, an equine, a bovine, an ovine, or a porcine.

In another aspect, the present invention provides methods of manufacturing a composition. The methods involve contacting a proton-donating (or “acidic”) pharmacologically active ingredient and a proton-accepting (or “basic”) pharmacologically active ingredient to provide a salt. In one embodiment, the methods further involves contacting the proton-donating pharmacologically active ingredient and the proton-accepting pharmacologically active ingredient in a solvent. In one embodiment, the solvent is a pharmaceutically acceptable solvent. Solid forms are obtained by simply evaporating the solvent to provide a solid dosage form. Representative solvents include, but are not limited to, glycerol formal, propylene glycol, N-methyl pyrollidone, dimethylsulfoxide, dimethyl acetamide, and polyethylene glycol. The proton-donating, pharmacologically active ingredient and the proton-accepting, pharmacologically active ingredient can be any proton donating and proton accepting pharmacologically active compounds, for example, those specified herein. The composition formed by the methods can be any specified herein.

The summary of the invention described above is not limiting and other features and advantages of the invention will be apparent from the following detailed description of the preferred embodiments, as well as from the claims.

### BRIEF DESCRIPTION OF THE DRAWINGS

FIG. 1 provides a graphical illustration of the in vitro release kinetics for a composition comprising tilmicosin and flunixin in a ratio of 1:2. (♦) represent the percent flunixin released and (□) represents the percent tilmicosin released.

FIG. 2 provides a graphical illustration of the in vitro release kinetics for a composition comprising tilmicosin, flunixin and lauric acid in a ratio of 1:1:1 for a formulation comprising 20 weight percent tilmicosin and 20 weight percent flunixin, wherein (□) and (°) represent the release rate of tilmicosin and flunixin, respectively and from a formulation comprising 10 weight percent tilmicosin and 10 weight percent flunixin, wherein (▴) and (∘) represent the release rate of tilmicosin and flunixin, respectively.

FIG. 3 illustrates the structural formula of oytetracycline.

FIG. 4 illustrates the structural formula of doxycycline.

FIG. 5 illustrates the structural formula of carprofen.

FIG. 6 illustrates the structural formula of flunixin.

FIG. 7 illustrates the structural formula of naproxen.

FIG. 8 illustrates the structural formula of tilmicosin.

FIG. 9 illustrates the structural formula of roxithromycin.

FIG. 10 illustrates the structural formula of azithromycin.

FIG. 11 illustrates the structural formula of terbinafine.

FIG. 12 is a plot of plasma concentration of flunixin (♦) and tilmicosin (▪) as a function of time when the formulation of Example 5a was administered to a foal as two 10 mL injections on each side of the neck on day 1 followed by two 10 mL injections on each side of the pectorals on day 7.

FIG. 13 are radiographs of the lungs of a foal suffering from Rhodococcus equi before treatment (FIG. 13a) and after treatment (FIG. 13b) with the formulation of Example 5b as described in Example 12.

FIG. 14 is a plot of plasma concentration of flunixin (♦) and tilmicosin (▪) as a function of time when the 1:1:1 Tilmicosin:Flunixin:Decanoic acid of Example 11 was administered to dogs at a tilmicosin dose of 10 mg/kg and a flunixin dose of 8 mg/kg. Each time point represents the average value for the plasma concentration of flunixin or tilmicosin of four dogs.

FIG. 15 is a plot of plasma concentration of flunixin as a function of time when commercially available flunixin ((Flunixamine®, commercially available from Phoenix Scientific, Inc. of St. Joseph, Mo.) is administered to dogs as a single dose of 1 mg/kg.

Each time point represents the average value for the plasma concentration of flunixin of two dogs.

### DETAILED DESCRIPTION OF THE PREFERRED EMBODIMENTS

The present invention relates to compositions comprising a salt formed from two or more pharmacologically active ingredients wherein at least one of the pharmacologically active ingredients is a proton donor (i.e., acidic) and the other pharmacologically active ingredients is a proton acceptor (i.e., basic). The invention further relates to methods of administering pharmacologically active ingredients and methods of treating a disorder in an animal comprising administering to an animal in need thereof a salt of the invention. The term “condition,” as used herein, means an interruption, cessation, or disorder of a bodily function, system, or organ, and includes diseases, defects, and disorders. Representative conditions include, but are not limited to, infections such as bacterial, viral, fungal, yeast, and parasitic infections; diseases such as cancer; inflammation; diabetes; and organ failure. In one embodiment, the salt is formed by combining a proton donating (or “acidic”) anti-inflammatory (e.g., flunixin, carprofen and naproxen) with a proton accepting (or “basic”) antibiotic (e.g., azithromycin, roxythromycin, tilmicosin, oxytetracycline and doxycycline). The two pharmacologically active ingredients form a neutral salt with a net charge of zero. In one embodiment, the salts are formed without any chemical modification to the structure of the pharmacologically active ingredient, other than formation of the salt. The salts disclosed herein can be provided in pharmacologically acceptable solvents, such as water miscible organic solvents. For example, the water miscible solvent can be pyrrolidone, N-methyl pyrrolidone, polyethylene glycol, propylene glycol (e.g., at about 10% in glycerol formal with or without stabilizers), glycerol formal, isosorbide dimethyl ether, ethanol, dimethyl sulfoxide, tetrahydrofurfuryl alcohol, triacetin, or any combination of these in any combined proportions, or another solvent found to have similar acceptable properties such as being non-toxic and soluble in water. The solvent can also be a water immiscible solvent. For example, the water immiscible solvent can be isopropyl myristate, ethyl lactate, castor oil, safflower oil, soybean oil, saw flower oil, castor oil, cottonseed oil, corn oil, sunflower oil, arachis oil, olive oil, a medium or long chain fatty acid, ethyl oleate, linoleic acid, isopropyl palmitate, a glycerol ester, a polyoxyl hydrogenated castor oil, cod liver oil, a fish derived oil, coconut oil, or combinations thereof. Other water immiscible solvents can also be identified that will find use in the present invention. In one embodiment, the mixture of active compound and water immiscible solvent forms a clear, true solution at room temperature.

In one embodiment, the combination of the salt and solvent result in a true injectable solution, but suspensions and other means of delivery are contemplated such as, for example, topical, oral or nasal delivery.

In other embodiments of the invention, additional counter-ions are present in the composition to regulate the release kinetics of the pharmacologically active ingredients from a dosage form comprising the salt of the invention. In the case of a salt of tilmicosin and flunixin, two molar equivalents of flunixin form a salt with 1 molar equivalent of tilmicosin. Yet in some embodiments, it may be desirable to administer more/less flunixin compared to tilmicosin. Thus, the compositions can include additional counter-ions to substitute for the proton donating and/or proton accepting pharmacologically active ingredients. The additional counter-ions can be lipophilic counterions, such as anions of fatty acids, as described above. Where it is desired to administer less of the acidic pharmacologically active ingredient, the fatty acid is included in an amount necessary to substitute for the acidic pharmacologically active ingredient. Thus, where the amount of flunixin in a tilmicosin-flunixin salt is sought to be reduced, an amount of fatty acid is supplied in the composition as a substitute for the appropriate amount of flunixin.

In still other embodiments of the invention, a third proton donating or proton accepting pharmacologically active ingredient is provided in the composition. The third pharmacologically active ingredient can form a salt with the other pharmacologically active ingredients in the composition. For example, if two equivalents of flunixin are required to one equivalent of tilmicosin, one of the equivalents of flunixin (or any portion thereof) can be replaced with another proton-donating pharmacologically active ingredient (e.g., carprofen or naproxen). Thus, the salts formed will include a salt of flunixin and tilmicosin and the salt of a second proton donating pharmacologically active ingredient (e.g., carprofen or naproxen) and tilmicosin. Any combination of proton donating and proton accepting pharmacologically active ingredients can be provided in the compositions to achieve the desired effect. Fatty acids or other lipophilic counterions can also be substituted, as described above, where no additional pharmacologically active compound is desired.

In yet other embodiments, excess pharmacologically active ingredients can be provided to supply an initial “burst” of active in the treated animal. Thus, where a salt of tilmicosin and flunixin is provided and an initial burst in the concentration of flunixin is desired, a molar excess of flunixin is provided in the amount desired for the initial burst. Thus, tilmicosin/flunixin salt will be administered to form a drug depot in the tissues of the treated animal but a quantity of the free form of flunixin will be immediately available to the system of the treated animal and form the initial burst concentration of flunixin.

Without wanting to be bound by any particular theory, it is believed that when solutions of the salt of a proton donating pharmacologically active ingredient and a proton accepting pharmacologically active ingredient are dissolved or suspended in a pharmaceutically acceptable solvent (e.g., a water-miscible solvent) and injected into a patient,.the soluble solvent diffuses away from the injection site resulting in the formation of a drug depot containing the salt. The proton donating and proton accepting pharmacologically active ingredients are ordinarily present in a solution in an equilibrium state wherein there is always some of the pharmacologically active ingredients present as the salt and some present in the free form. Over time, the salt re-equilibrates with appropriate counter ions in the animal's body and both pharmacologically active ingredients are released in a controlled, time-release manner.

Several advantages are obtained with the present invention. For example, as shown below, it was demonstrated that a single dose injection of a tilmicosin-flunixin salt prepared in accordance with the present disclosure provided pharmaceutically effective amounts of the anti-inflammatory flunixin which was present in the blood or tissues of a treated animal for a period of days, such as up to 4 days or up to 5 days or up to 6 days. A flunixin injection administered according to previous methods is normally present in tissues at a pharmaceutically effective amount for only 6-12 hours when administered in a conventional format. In other embodiments, other pharmacologically active compounds are present in the blood or tissues of the animal at pharmaceutically effective amounts for different periods of time, such as up to 4 days, up to 6 days, up to 8 days, up to 10 days or up to 12 days or up to 15 days or up to 18 days or up to 20 days or up to 25 days or up to 30 days, depending on the particular pharmacologically active compound.

The toxicity of pharmacologically active compounds is also reduced when administered according to the present invention. Example 11 below shows that when a dog was subcutaneously injected with three repeated doses of a tilmicosin-flunixin salt (prepared in accordance with the present disclosure) at 8 mg/kg with a week interval (i.e., one dose a week for three weeks), there was no observed toxicity or injection site reactions. Complete necropsy of the dog further demonstrated no deleterious effects on any of the organs, including the gastrointestinal track. Also, as stated above, flunixin injections and tilmicosin injections have been known to cause injection site reactions. This is not the case with a tilmicosin-flunixin salt prepared and administered according to the present invention.

The following examples illustrate how various compositions of the invention were successfully prepared. These examples are merely illustrative and are not intended to be limiting. With reference to the present disclosure the person of ordinary skill in the art will realize that many different compositions can be formed using the methods, and these are also encompassed by the scope of the present application.

### EXAMPLES

### Example 1

### Azithromycin-Flunixin Combination

This example illustrates how a composition of the invention was prepared containing azithromycin and flunixin. 20 grams of azithromycin dihydrate and 15.46 grams of flunixin were weighed in to a 100 mL volumetric flask. To these solids was added 5 mL of propylene glycol followed by the addition of stabilized glycerol formal to 75% volume. The flask was sonicated for about 15 min and left on a shaker until a clear solution was obtained. Finally, the volume was made up to 100 mL with stabilized glycerol formal and mixed well to obtain a homogeneous solution.

### Example 2

### Roxythromycin-Flunixin Combination

This example illustrates how a composition of the invention containing roxythromycin and flunixin was prepared. 5 grams of roxithromycin and 2.16 grams of flunixin were weighed in to a 25 mL volumetric flask. To these solids was added 1.25 mL of propylene glycol followed by the addition of stabilized glycerol formal to 75% volume. The flask was sonicated for about 15 min and left on a shaker until a clear solution was obtained. Finally, the volume was made up to 25 mL with stabilized glycerol formal and mixed well to obtain a homogeneous solution.

### Example 3

### Oxytetracycline-Flunixin Combination

This example illustrates how a composition of the invention containing oxytetracycline and flunixin was prepared. 7.5 grams of oxytetracycline and 4.822 grams of flunixin were weighed in to a 50 mL volumetric flask. To these solids was added N-methylpyrrolidone (NMP) to 90% volume. The flask was sonicated for about 15 min and left on a shaker for another 30 minutes to obtain a clear solution. Finally, the volume was made up to 50 mL with NMP and mixed well to obtain a homogeneous solution.

### Example 4

### Doxycycline-Flunixin Combination

7.5 grams of oxytetracycline and 4.8 grams of flunixin were weighed in to a 50 mL volumetric flask. To these solids was added N-methylpyrrolidone (NMP) to 90% volume. The flask was sonicated for about 15 min and left on a shaker for another 30 minutes to obtain a clear solution. Finally, the volume was made up to 50 mL with NMP and mixed well to obtain a homogeneous solution.

### Example 5

### Tilmicosin-Flunixin Combination

Method A: 7.5 grams of tilmicosin and 5.37 grams of flunixin were weighed in to a 50 mL volumetric flask. To these solids was added 2.5 mL of propylene glycol followed by the addition of stabilized glycerol formal to 75% volume. The flask was sonicated for about 15 min and left on a shaker until a clear solution was obtained. Finally, the volume was made up to 50 mL with stabilized glycerol formal and mixed well to obtain a homogeneous solution.

Method B: 10 grams of tilmicosin and 7.161 grams of flunixin were weighed into a 50 mL volumetric flask. To these solids was added NMP to 75% volume. The flask was sonicated for about 15 min and left on a shaker until a clear solution was obtained. Finally, the volume was made up to 50 mL with NMP and mixed well to obtain a homogeneous solution.

### Example 6

### Tilmicosin-Carprofen-Linoleic Fatty Acid Combination

7.5 grams of tilmicosin, 2.362 grams of carprofen and 2.54 grams of linoleic acid were weighed in to a 50 mL volumetric flask. To this mixture was added 2.5 mL of propylene glycol followed by the addition of stabilized glycerol formal to 75% volume. The flask was sonicated for about 15 min and left on a shaker until a clear solution was obtained. Finally, the volume was made up to 50 mL with stabilized glycerol formal and mixed well to obtain a homogeneous solution.

### Example 7

### Tilmicosin-Flunixin-Fatty acid Combination

7.5 grams of tilmicosin, 2.56 grams of flunixin and 2.54 grams of linoleic acid were weighed in to a 50 mL volumetric flask. To this mixture was added 2.5 mL of propylene glycol followed by the addition of stabilized glycerol formal to 75% volume. The flask was sonicated for about 15 min and left on a shaker until a clear solution was obtained. Finally, the volume was made up to 50 mL with stabilized glycerol formal and mixed well to obtain a homogeneous solution.

### Example 8

### Tilmicosin-Naproxen Combination

7.5 grams of tilmicosin and 4.17 grams of naproxen were weighed in to a 50 mL volumetric flask. To these solids was added N-methylpyrrolidone (NMP) to 90% volume. The flask was sonicated for about 15 min and left on a shaker for another 30 minutes to obtain a clear solution. Finally, the volume was made up to 50 mL with NMP and mixed well to obtain a homogeneous solution.

### Example 9

### Terbinafine-Flunixin Combination

7.5 grams of terbinafine and 8.0 grams of flunixin were weighed in to a 50 mL volumetric flask. To these solids was added N-methylpyrrolidone (NMP) to 90% volume. The flask was sonicated for about 15 min and left on a shaker for another 30 minutes to obtain a clear solution. Finally, the volume was made up to 50 mL with NMP and mixed well to obtain a homogeneous solution.

### Example 10

### Tilmicosin-Flunuxin-Decanoic Fatty Acid Acid Composition

82.97 grams of tilmicosin, 53.7 grams of flunixin, and 31.23 grams of decanoic acid were weighed into a clean and dry 500 mL volumetric flask. 25 mL of propylene glycol was pipetted into the flask followed by making up 75% of the volume with glycerol formal. The flask was placed on a shaker with occasional sonicating for about 30 min to provide a clear solution. The flask was then filled to 500 mL with glycerol formal.

### Example 11

### Modulation of Release Kinetics

The release kinetics of the pharmacologically active ingredients can be modulated by changing the following variables. One variable is the ratio of the proton donating pharmacologically active ingredient to the proton accepting pharmacologically active and fatty acid that is substituted in part for the proton donating pharmacologically active ingredient, if such substitution is made. In this example, the tilmicosin is basic and the flunixin is acidic. Two equivalents of flunixin is required to neutralize tilmicosin, which has 2 basic tertiary nitrogens. A fatty acid can be used in part to substitute for flunixin as the proton-donating component in the salt formation to thereby influence the in vitro release kinetics. Two formulations were made, using tilmicosin:flunixin:lauric acid at 1:1:1 and 1:2:0 ratios. The rate of release of tilmicosin and flunixin as a function of time was determined by placing 1 mL aliquots of each of the resulting formulations in sealed dialysis bags and then suspending the dialysis bags in flasks containing 150 mL of phosphate-buffered saline at pH 7.4. A precipitate was observed to form in the dialysis bag within about 1 hour. Aliquots of saline were then removed at various intervals and the concentration of tilmicosin in the saline was determined using high pressure liquid chromatography (HPLC).

For HPLC analysis 100 μL was injected on a Phenomenex Luna 5 μM phenyl-hexyl 100A, 250×4.6 mm analytical column operated at a flow rate of 1.7 mL/min. The HPLC was interfaced to a UV detector operated at 285 nm. The HPLC column was eluted using gradient elution according to the following profile:

| Time | Percent Pump A | Percent Pump B |
|---|---|---|
| 0 | 30 | 70 |
| 10.5 | 85 | 15 |

wherein the solvent in pump A was 25 mM phosphate buffer at pH 2.4 and the solvent in pump B was acetonitrile. The total run time was 25 min. The serum concentration of flunixin and tilmicosin was then determined by comparing the area under the curve for the HPLC peak corresponding to flunixin or tilmicosin to a standard curve of peak areas v. known concentrations of flunixin or tilmicosin in phosphate-buffered saline. The standard curve was prepared using the following concentrations of flunixin and tilmicosin 4, 2, 1, 0.5, and 0 μg/mL.

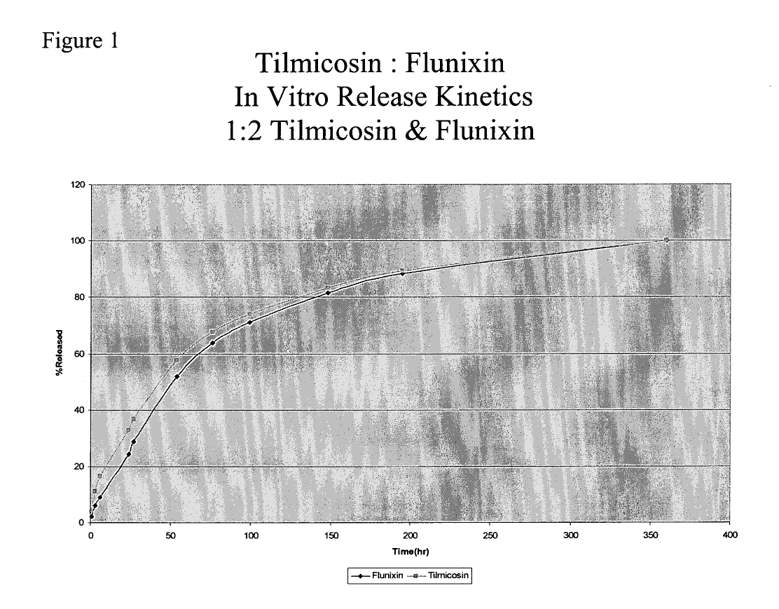

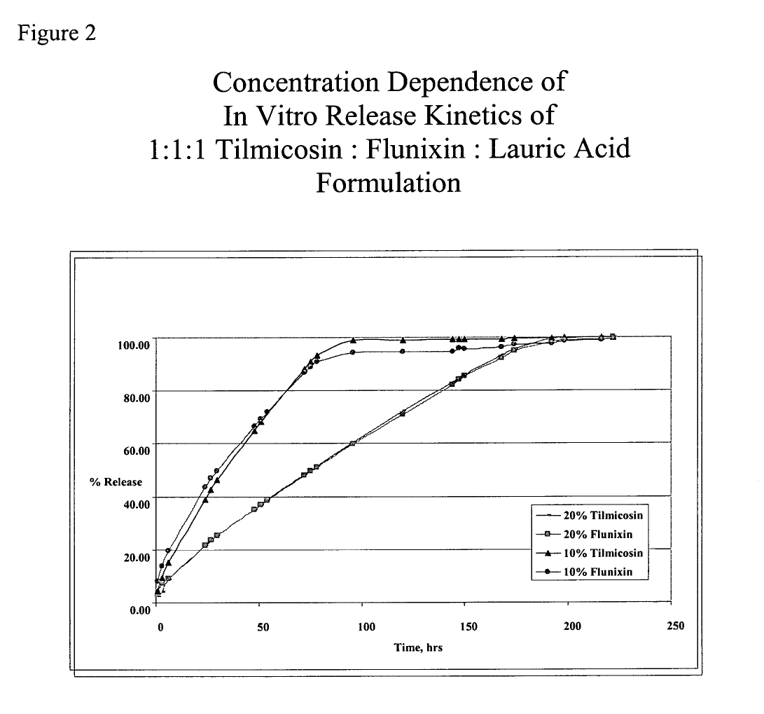

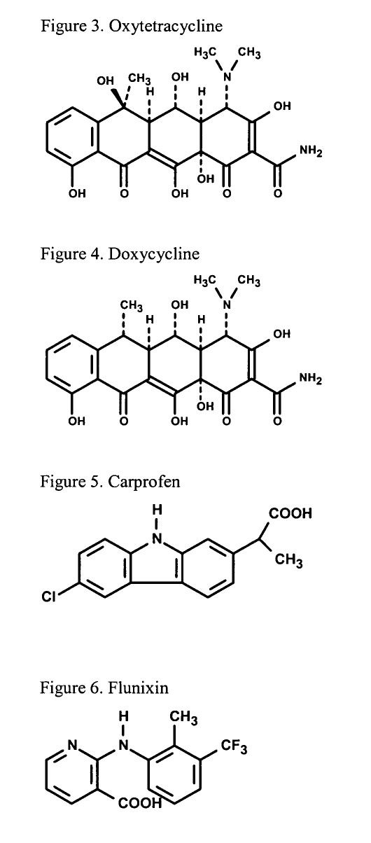

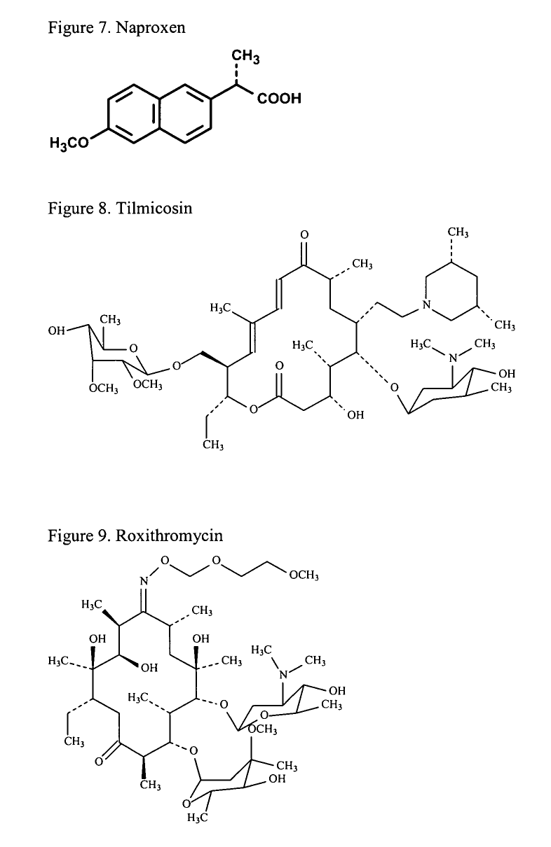

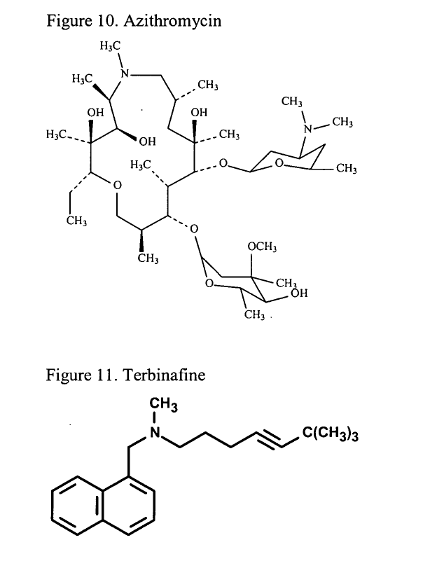

As demonstrated in FIGS. 1 and 2, the results suggested that the formulation containing fatty acid partly substituted for one pharmacologically active ingredient releases the pharmacologically active ingredient faster than the one without fatty acid.

The hydrophobic carbon chain length of the fatty acid used is another variable that can be used to modulate the release kinetics. Fatty acids such as decanoic, lauric, linoleic acids, and others find use in the invention. Longer chain lengths of the fatty acid correlate with a slower release kinetics. Thus, a linoleic acid having 18 carbons will have slower release kinetics than a lauric acid having 12 carbons.

Other variables that can be used to modulate the release kinetics of the pharmacologically active ingredients include the pharmaceutically acceptable solvent used, and the concentration of the formulation.

### Example 12

### In vivo Study in Dogs

A dog was subcutaneously injected in three phases at a dose of 8 mg/kg with a formulation of tilmicosin-flunixin salt (1:2 ratio) prepared according to the present invention (see Example 5a). A one week interval was used between phases and serum samples were collected to assay for tilmicosin and flunixin by HPLC.

Blood samples were treated and analyzed for tilmicosin and flunixin according to the following procedure:
(i) A Strata X-C 33 μm Cation Mixed-Mode Polymer 30 mg/mL cartridge was condition by washing with 1 mL of methanol and 1 mL of deionized water using gravity flow;
(ii) 1 mL of serum acidified with 20 μl of phosphoric acid was applied to the conditioned cartridge;
(iii) The column was washed with 1 mL of 0.1% H₃PO₄/H₂O, 1 mL of acetonitrile, and 2 mL of methanol;
(iv) The column was eluted with 4 mL ammonia in methanol (15% of 2M NH₄OH in methanol);
(v) The solvent was removed from the eluant using a stream of nitrogen gas; and
(vi) The resulting residue was then reconstituted with 1 mL of 50:50 methanol/50 mM phosphate buffer at pH 2.3 and analyzed by HPLC using the HPLC method described in Example 11.

Analysis of the serum for flunixin and tilmicosin is presented in Table 1.

TABLE 1
Tilmicosin-Flunixin Serum Data, Dog Study
(μg/ml)

| Time | Phase 1 | | Phase 2 | | Phase 3 | |
|---|---|---|---|---|---|---|
| (hrs) | Flunixin | Tilmicosin | Flunixin | Tilmicosin | Flunixin | Tilmicosin |
| 6 | 1.86 | 0.16 | 3.7 | 0.19 | 4.01 | 0.23 |
| 12 | 1.69 | 0.21 | 3.4 | 0.24 | 3.12 | 0.21 |
| 24 | 2.77 | 0.15 | 3.47 | 0.21 | 4.7 | 0.19 |
| 48 | 2.13 | 0.18 | 1.81 | 0.2 | 2.1 | 0.15 |
| 72 | 1.58 | 0.28 | 1.15 | 0.4 | 0.4 | 0.09 |
| 96 | 1.03 | 0.19 | 0.04 | 0.16 | 0.29 | 0 |
| 120 | 0.02 | 0.14 | 0 | 0.22 | 0.22 | 0 |
| 144 | 0.0 | 0.0 | 0 | 0.0 | 0.0 | 0 |

On day 28, the animal was sacrificed and a complete necropsy was performed to determine whether the large dose of flunixin had caused any deleterious effects on the gastro-intestinal tract or other organs, such as the liver, kidney, lungs and heart. The analysis of the serum samples suggested that the flunixin was released over a period of 4 days at physiologically relevant concentrations. The necropsy results showed no indication of any deleterious effects on any of the organs examined, including the gastro-intestinal tract. Furthermore, no significant reaction was observed at the site of injection.

### Example 13

### In vivo Study in a Foal

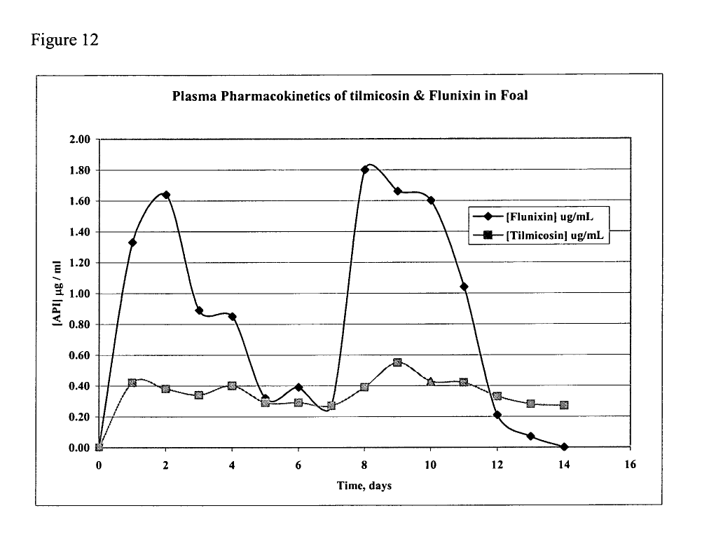

A 300 pound foal suffering from Rhodococcus equi infection was treated with a combination of timicosin and flunixin. On day one, the foal was administered two 10 mL injections of the tilmicosin/flunixin formulation of Example 5a, one subcutaneous injection on each side of the neck (total injection volume 20 mL). On day seven another 20 mL dose was administered as a 10 mL subcutaneous injection on each side of the pectorals. This is equivalent to 10 mg/kg of tilmicosin and 7.16 mg/kg of flunixin per dose. Blood samples were drawn at different time points and analyzed for both tilmicosin and flunixin. Blood samples were treated and analyzed as described in Example 12. Concentrations of tilmicosin and flunixin in the blood as a function of time are presented in FIG. 12. The data presented in FIG. 12 show that the tilmicosin and flunixin are released in a controlled manner over a period of 7 days following each administration.

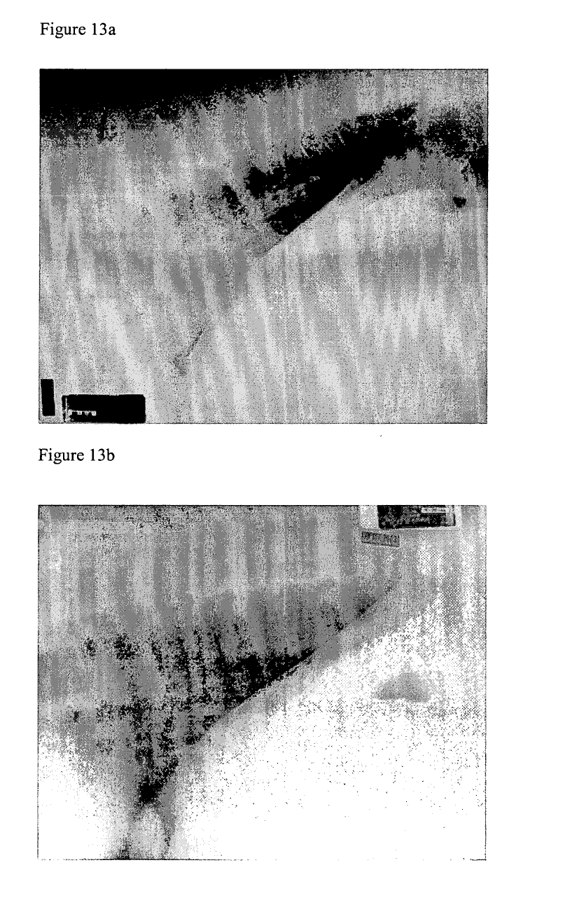

Radiographs of the foals lungs before treatment (FIG. 13a) and after treatment (FIG. 13b) show that foal responded to treatment.

### Example 14

### In vivo Study in Dogs

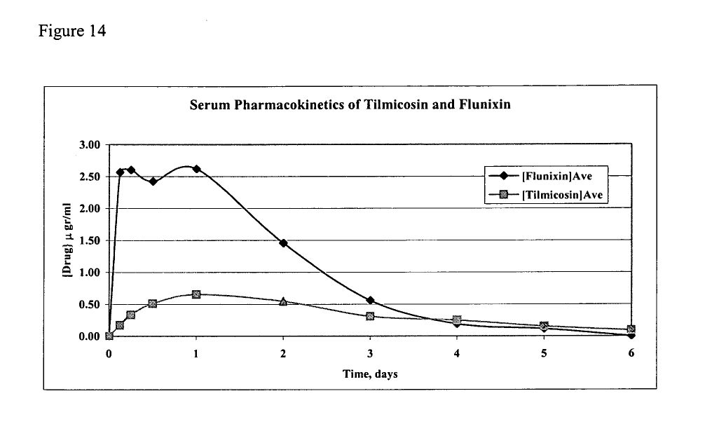

Four dogs were each injected with a formulation of 1:1:1 Tilmicosin:Flunixin:Decanoic acid (prepared as described in Example 10) to provide a tilmicosin dose of 11.2 mg/kg and flunixin dose of 8 mg/kg. Blood samples were drawn at different time points and analyzed for both tilmicosin and flunixin. Blood samples were treated and analyzed as described in Example 12. Concentrations of tilmicosin and flunixin in the blood as a function of time are presented in FIG. 14. The data presented in FIG. 14 show that the tilmicosin and flunixin are release over time in a controlled manner at physiologically significant levels for between 3 and 4 days.

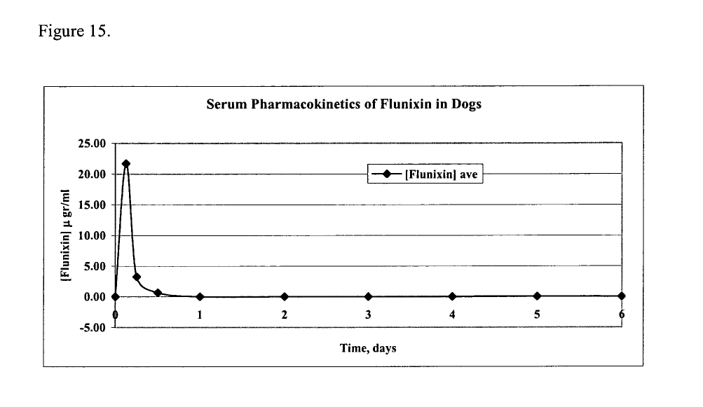

In contrast, administering a single dose of 1 mg/kg of commercially available flunixin (Flunixamine®, commercially available from Phoenix Scientific, Inc.) to dogs by subcutaneous injection at a dose of 8 mg/kg results in a C_{Max} of 21.5 μg/mL in 3 h that then rapidly drops to sub-microgram/mL after 6 h and is undetectable in the blood 12 hours after injection. A plot of flunixin concentration in the blood vs. time is provided in FIG. 15.

While the invention has been described and exemplified in sufficient detail for those skilled in this art to make and use it, various alternatives, modifications, and improvements should be apparent without departing from the spirit and scope of the invention.

One skilled in the art will readily appreciate that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are set forth within the following claims.

1. A composition comprising a salt of a proton-donating pharmacologically active ingredient and a proton-accepting pharmacologically active ingredient.

2. The composition of claim 1, wherein the proton-donating, pharmacologically active ingredient has anti-inflammatory activity.

3. The composition of claim 2 wherein the proton-donating, pharmacologically active ingredient is a non-steroidal anti-inflammatory (NSAID).

4. The composition of claim 3, wherein the NSAID is selected from the group consisting of: flunixin, carprofen, ibuprofen, diclofenac, and naproxen.

5. The composition of claim 4, wherein the NSAID is flunixin.

6. The composition of claim 1, wherein the proton-accepting pharmacologically active ingredient has anti-infective or anti-microbial activity.

7. The composition of claim 6, wherein the proton-accepting pharmacologically active ingredient is an antibiotic.

8. The composition of claim 7, wherein the proton-accepting pharmacologically active ingredient is selected from the group consisting of azithromycin, roxythromycin, tilmicosin, oxytetracycline and doxycycline.

9. The composition of claim 8, wherein the proton-accepting pharmacologically active ingredient is tilmicosin.

10. The composition of claim 1 wherein the proton-donating pharmacologically active ingredient is selected from the group consisting of flunixin, carprofen, and naproxen; and
the proton-accepting pharmacologically active ingredient is selected from the group consisting of azithromycin, roxythromycin, tilmicosin, oxytetracycline and doxycycline.

11. The composition of claim 1, wherein the proton-donating pharmacologically active ingredient is flunixin and the proton-accepting active ingredient is tilmicosin.

12. The composition of claim 1 further comprising a pharmaceutically acceptable carrier, and wherein the composition is an injectable composition that forms a precipitate when injected into water.

13. The composition of claim 12, wherein the injectable composition is a solution.

14. The composition of claim 1, wherein at least one of the pharmacologically active ingredients is a COX-2 inhibitor.

15. The composition of claim 14, wherein the COX-2 inhibitor is celecoxib.

16. The composition of claim 1, wherein the proton donating or proton accepting pharmacologically active ingredient is selected from the group consisting of a macrolide, a tetracycline, an aminoglycoside, a β-lactam, and an antifungal.

17. The composition of claim 1, further comprising a salt formed from a proton-accepting pharmacologically active ingredient and a proton-donating lipophilic molecule.

18. The composition of claim 17, wherein the lipophilic molecule is a fatty acid.

19. The composition of claim 18, wherein the fatty acid is selected from the group consisting of lauric acid, linoleic acid, decanoic acid, myristic acid, and oleic acid.

20. The composition of claim 1, further comprising a pharmacologically active ingredient in free form.

21. A method of administering a pharmacologically active ingredient to an animal comprising administering to the animal a composition comprising (i) a salt of a proton donating pharmacologically active ingredient and a proton accepting pharmacologically active ingredient and (ii) a pharmaceutically acceptable carrier.

22. The method of claim 21, wherein:
the proton-donating pharmacologically active ingredient is selected from the group consisting of flunixin, carprofen, and naproxen; and
the proton accepting pharmacologically active ingredient is selected from the group consisting of azithromycin, roxythromycin, tilmicosin, oxytetracycline and doxycycline.

23. The method of claim 22, wherein the composition is administered by injection.

24. The method of claim 22, wherein the proton-donating pharmacologically active ingredient and the proton-accepting, pharmacologically active ingredient have slower release kinetics in the animal when administered as the salt than when administered in free form.

25. The method of claim 22, wherein the animal is selected from the group consisting of a human, a canine, a feline, an equine, a bovine, an ovine, or a porcine.

26. A method of manufacturing a composition comprising contacting a proton donating pharmacologically active ingredient and a proton accepting pharmacologically active ingredient.

27. The method of claim 26, wherein the proton donating pharmacologically active ingredient and a proton accepting pharmacologically active ingredient are contacted in a solvent.

28. The method of claim 26, wherein the proton-donating pharmacologically active ingredient has anti-inflammatory activity.

29. The method of claim 28, wherein the proton-donating pharmacologically active compound is a non-steroidal anti-inflammatory (NSAID).

30. The method of claim 26, wherein the proton-donating pharmacologically active ingredient is selected from the group consisting of flunixin, carprofen, and naproxen; and
the proton-accepting pharmacologically active ingredient is selected from the group consisting of azithromycin, roxythromycin, tilmicosin, oxytetracycline and doxycycline.

31. The method of claim 30, wherein the proton-donating pharmacologically active ingredient is flunixin and the proton-accepting pharmacologically active ingredient is tilmicosin.

32. An injectable composition comprising about 10 to 30 percent by weight of tilmicosin, about 2 equivalents of flunixin per equivalent of tilmicosin, and about 10 percent propylene glycol in glycerol formal.

33. An injectable composition comprising about 10 to 30 percent by weight of tilmicosin, about 1 equivalent of flunixin per equivalent of tilmicosin, about 1 equivalent of a fatty acid per equivalent of tilmicosin, and about 10 percent propylene glycol in glycerol formal.

34. The composition of claim 33, wherein the fatty acid is decanoic acid or lauric acid.
